(19) United States
(12) Patent Application Publication
(10) Pub. No.: US 2025/0059111 A1
(43) Pub. Date: Feb. 20, 2025

(54) PROCESS FOR CONVERTING DIMETHYL ETHER OR METHANOL TO HYDROCARBONS LOW IN AROMATIC COMPOUNDS, USING A PALLADIUM-LOADED ZEOLITE CATALYST

(71) Applicant: KARLSRUHER INSTITUT FUER TECHNOLOGIE, KARLSRUHE (DE)

(72) Inventors: BENJAMIN NIETHAMMER, STUTTGART (DE); ULRICH ARNOLD, BRUCHSAL (DE); JOERG SAUER, KARLSRUHE (DE)

(73) Assignee: KARLSRUHER INSTITUT FUER TECHNOLOGIE, KARLSRUHE (DE)

(21) Appl. No.: 18/721,201

(22) PCT Filed: Nov. 23, 2022
(86) PCT No.: PCT/EP2022/082930

§ 371 Date: Jun. 18, 2024

(30) Foreign Application Priority Data
Dec. 20, 2021 (DE) 10 2021 133 788.7

(51) Int. Cl. C07C 1/20; B01J 29/74; B01J 35/40; B01J 37/02; B01J 37/08

(52) U.S. Cl. CPC C07C 1/20; B01J 29/7461; B01J 35/40; B01J 37/0201; B01J 37/08; C07C 2529/74

(57) ABSTRACT

The present invention provides a method which includes loading a zeolite catalyst in ammonium form with palladium ions, drying and calcining the palladium ion-laden zeolite catalyst to a proton form, fractionating the proton form of the palladium ion-laden zeolite catalyst into a powder, mixing the powder with an inert material, introducing the mixture into a fixed bed reactor, heating the fixed bed reactor to a reaction temperature and passing an inert gas therethrough, reducing the mixture in a hydrogen stream, passing an inert gas through the fixed bed reactor, introducing a reactant gas feed of hydrogen, an inert gas and dimethyl ether or methanol into the fixed bed reactor to form a product gas mixture, condensing the product gas mixture to a product phase liquid, and separating the product phase liquid into an aqueous and into an organic phase which includes the low-aromatics C₅₊ hydrocarbons.

### CROSS REFERENCE TO PRIOR APPLICATIONS

This application is a U.S. National Phase application under 35 U.S.C. § 371 of International Application No. PCT/EP2022/082930, filed on Nov. 23, 2022 and which claims benefit to German Patent Application No. 10 2021 133 788.7, filed on Dec. 20, 2021. The International Application was published in German on Jun. 29, 2023 as WO 2023/117271 A1 under PCT Article 21(2).

### FIELD

The present invention relates to a process for converting dimethyl ether (DME) or methanol into low-aromatics hydrocarbons using a palladium-laden zeolite catalyst.

Liquid hydrocarbon fuels play an essential role in the global energy supply chain due to their high energy density and easy transportability. The conversion of methanol or DME in so-called methanol-to-gasoline (MTG) or DME-to-gasoline (DTG) processes allows for the production of high-quality synthetic fuels and represents a key step for obtaining synthetic gasolines from both fossil and renewable raw materials. The MTG process from ExxonMobil developed in the 1970s makes it possible to convert methanol or DME into hydrocarbons in the boiling range of gasoline via the zeolite catalyst “ZSM-5”.

Zeolites are three-dimensional networks that are typically formed from SiO₄ and AlO₄ tetrahedra joined via oxygen bridges which contain both water and freely movable alkali metal cations for charge compensation. The general empirical formula for zeolites is:

Mⁿ⁺_{x/n}[(AlO₂)⁻ₓ(SiO₂)_{y}]·z H₂O.

The factor n is the charge of the cation M and is usually 1 or 2. M is typically a cation of an alkali metal or alkaline earth metal. The factor z indicates how many water molecules have been absorbed by the crystal. Depending on the structure type, the zeolites exhibit a structure of uniform pores and/or channels in which substances can be adsorbed. These are known as micropores or mesopores depending on pore size. Such materials have an extraordinarily large internal surface area of well over 1000 m²·g⁻¹ in some cases. Through selection of the zeolite, it is possible to control the pore diameter so that only certain reactant molecules can pass through the pores to the catalytically active centers in the interior or only certain products can again exit the zeolite. This is referred to as reactant or product selectivity. The size of the interior cavity can also be controlled so that only certain transition states are possible and thus only certain products can be formed. Aluminium-containing zeolites have a negative framework charge due to the trivalent aluminum atoms, each of which can formally be assigned two divalent oxygen atoms. Cations are therefore present at the inner and outer surface of aluminum-containing zeolites, wherein, in water-containing zeolites, the cations are often present in the channel systems of the zeolites in dissolved form and are thus relatively easily accessible and exchangeable. The typical cations Na⁺, K⁺, Ca²⁺ and Mg²⁺ may be exchanged for ammonium ions by ion exchange (forming the ammonium form of the zeolite). Exchanging the free cations in the zeolites for protons forms solid-state acids, the so-called H-zeolites. The acidity of these H-zeolites may be adjusted by the degree of ion exchange or partial dealumination over a wide range.

It has recently also become possible to synthesize nanoscale zeolites, i.e., zeolite materials having particle diameters below 100 nanometers, which provide markedly improved transport properties compared to conventional zeolites. These improved properties are of great importance in catalysis and in adsorption processes in which zeolites are employed.

Zeolites are among the most important catalysts in the chemical industry due to their high shape selectivity, their adjustable acidic properties, their thermal stability and their capacity for regeneration. Either the zeolite itself acts as an acidic catalyst or introduced metal particles are the actual active centers. Metal-laden zeolites may also be used as bifunctional catalysts for multi-stage reactions.

In the course of the MTG process, methylation and oligomerization of light olefins results in higher olefins having five or more carbon atoms (C₅₊ olefins) which combine by various reaction paths to form acyclic saturated hydrocarbons of the general formula CₙH₂ₙ₊₂, cyclic saturated hydrocarbons, and methylated aromatics. The shape-selective zeolite catalyst ZSM-5 limits the hydrocarbon synthesis reactions to a chain length of about eleven carbon atoms.

In the original MTG process using the zeolite catalyst ZSM-5, the product mixture (containing gaseous, liquid and optionally solid components) is relatively rich in unsaturated hydrocarbons and aromatic components.

The presence of aromatics in the product mixture of the MTG process has a positive effect on the knock resistance of the fuels to be produced due to the high octane numbers, however, the aromatics contribute significantly to the formation of pollutants, especially soot particles, during combustion of the fuel. This demands capable and costly exhaust aftertreatment systems to meet increasingly stringent threshold values.

The knock resistance of low-aromatics gasolines is problematic. If aromatic components are avoided, the associated reduction in knock resistance must be compensated by other components. It has been found that highly branched acyclic saturated hydrocarbons exhibit a high knock resistance without further processing and without addition of octane-number enhancers.

A product mixture rich in aromatic components must be worked up for production of alternative, low-emission fuels, which entails additional process steps such as hydrogenation and isomerization reactions. This impairs not only the sustainability of the process, but also its efficiency and economy.

There is therefore a need for a modified MTG process which allows for the production of acyclic, branched hydrocarbons having a higher hydrogen content (empirical formulae CₙH₂ₙ₊₂ and CₙH₂ₙ) and thus overcomes the disadvantages associated with the production of aromatics-rich hydrocarbon mixtures. Saturated (CₙH₂ₙ₊₂) products in the C₅-C₁₁ chain length range may be directly employed as synthetic gasoline. Unsaturated, acyclic (CₙH₂ₙ) products offer a high level of flexibility for further processing by oligomerization, isomerization or hydrogenation, which is relatively easy to perform. A modified MTG process could thus generate a broad palette of end products, for example, gasoline, diesel or kerosene, adapted to the individual requirements of the producer.

Since the MTG reaction proceeds primarily within the zeolite crystals, the product selectivity of the reaction depends strongly on the size and dimensionality of the zeolite channel system. Mesoporous (10-ring) zeolites, such as ZSM-5 comprising three-dimensional channels having dimensions of 5.4×5.6 Å and 5.3×5.5 Å, typically have a high selectivity for hydrocarbons having five or more C atoms (C₅₊ hydrocarbons), wherein the product mixture of the MTG reaction contains primarily aromatics and paraffins.

Microporous zeolite catalysts of the structure type *MRE (https://europe.iza-structure.org/IZA-SC/material_tm.php?STC=MRE) have a one-dimensional 10-ring pore structure having dimensions of 5.3×5.6 Å with unidirectional, non-intersecting channels.

S. Teketel et al., ACS Catal. 2012, 2, 26-37 investigated the MTG process on the one-dimensional, non-intersecting 10-ring zeolites ZSM-22, ZSM-23, ZSM-48 and EU-1.

The influence of small differences in the channel systems of the materials (size and shape) on product distribution and stability under different reaction conditions was thereby tested. The influence of coke deposits on product selectivity in a fixed bed reactor at temperatures between 350° C. and 500° C. and a weight hourly space velocity (WHSV) between 2 and 6 g·g⁻¹·h⁻¹ was also investigated. With the exception of EU-1, all catalysts showed a high selectivity for low-aromatics C₅₊ hydrocarbons in the boiling range of gasoline fuels which was independent of the increasing coking of the zeolite catalyst during the reaction. Due to the relatively rapid deactivation of the zeolite catalysts ZSM-22, ZSM-23, ZSM-48 and EU-1, low feed rates and temperatures above 350° C. were essential for effective conversion via these catalysts. The one-dimensional pore system of the catalysts tested resulted in rapid catalyst deactivation through pore blocking. The cycle life of these one-dimensional zeolites therefore did not exceed 1-10 hours.

The stability of the catalysts during the MTG process was determined via the methanol conversion capacity which is defined as the total amount of methanol in grams converted to hydrocarbons per gram of catalyst before complete deactivation of the catalyst. For ZSM-48, the methanol conversion capacities were 15.8 gₘₑₜₕₐₙₒₗ·g_{catalyst}⁻¹ at 400° C. and 14.3 gₘₑₜₕₐₙₒₗ·g_{catalyst}⁻¹ at 450° C.

In contrast to the catalysts ZSM-22 and ZSM-23, the catalyst ZSM-48 showed a product spectrum that contained considerable amounts of aromatics. This observation could be attributed to the wider channels of the ZSM-48 catalyst compared to ZSM-22 and ZSM-23. The channel dimensions of ZSM-48, which are similar to the well-known ZSM-5 catalyst, enabled formation and diffusion of aromatic reaction products in the tested MTG process despite the lack of channel intersections in ZSM-48. The selectivity for C₅₊ hydrocarbons was higher for the ZSM-48 catalyst than for the ZSM-22 and ZSM-23 catalysts and amounted to about 55-75% over the product spectrum. The selectivity for aromatics for ZSM-48 was 20-40%; within this aromatics fraction, penta- and hexamethylbenzene, which have relatively low crystallization temperatures, were formed to a considerable extent using ZSM-48 (S. Teketel et al., ACS Catal. 2012, 2, 26-37; J. Li et al., Catalysis Today 2011, 164, 288-292).

US 2015/0353840 A1 describes the reaction of DME and/or methanol in the presence of hydrogen via metal-laden zeolite catalysts in a temperature range of 150-300° C. to afford C₄-C₉-hydrocarbons having a markedly reduced proportion of aromatic components and a high proportion of linear and branched paraffins and olefins. Small-, medium- or large-pored zeolite types may be used. In one embodiment of US 2015/0353840 A1, the zeolite catalyst contains a first active material deposited on a surface and/or in the pores of the zeolite composed of at least one of the elements copper, zinc, iron, gallium, lanthanum, platinum and/or mixtures thereof. The solid catalyst may also contain a second active material, wherein the second active material may be selected from the elements silver, copper, platinum, gallium, palladium and/or mixtures thereof.

CN 106867564 A describes a process for producing isoparaffin-rich gasoline from methanol and/or DME using a reducing gas and a catalyst which comprises a metal-active component having a mass fraction wᵢ in the range from wₘₑₜₐₗ=0.01-15% and a molecular sieve in the range from w_{zeolite}=85-99.99%. The mass fraction w; is defined as the value of the quotient of the mass mᵢ of the mixture component i under consideration and the sum of the masses of all components (including i) of the mixture (standard DIN 1310: composition of mixed phases). The produced hydrocarbons here have a carbon number from 5 to 11 with a low proportion of olefins and aromatic hydrocarbons. The reduction gas may be hydrogen or carbon monoxide, the metal-active component Cr, W, Fe, Co, Ni, Cu, Zn, Pd, Pt and Ga and the employed molecular sieve a zeolite from the group of H-ZSM-5, H-ZSM-22, H-beta or HY. For the reaction temperature, CN 106867564 A specifies a range from 250-450° C. and a pressure of 0.1-3.0 MPa (1-30 bar).

The hitherto-known MTG and DTG processes for producing low-aromatics hydrocarbons have the disadvantage that the employed zeolite catalysts exhibit relatively low reactant conversions, low space-time yields, and a low catalyst stability.

### SUMMARY

An aspect of the present invention is to provide low-aromatics hydrocarbons and a process for the production thereof using a zeolite catalyst, wherein high reactant conversions and a high space-time yield should be achieved, and the employed catalyst should have a high long-term stability. A further aspect of the present invention is to provide a use for the process.

In an embodiment, the present invention provides a method for converting dimethyl ether (DME) or methanol into low-aromatics C₅₊ hydrocarbons via a catalytic reaction over a palladium-laden zeolite catalyst H-EU-2 in a hydrogen stream. The method includes:
A) loading a EU-2 zeolite catalyst in an ammonium form with palladium ions via a capillary impregnation to obtain a palladium ion-laden zeolite catalyst;
B) drying and calcining the palladium ion-laden zeolite catalyst from step A) to form a proton form of the palladium ion-laden zeolite catalyst;
C) fractionating the proton form of the palladium ion-laden zeolite catalyst from step B) into a powder having a particle size of 100 to 500 μm;
D) mixing the powder from step C) with an inert material to obtain a mixture;
E) introducing the mixture from step D) into a fixed bed reactor;
F) heating the fixed bed reactor from step E) to a reaction temperature of 350 to 450° C. and passing a flow of an inert gas through the fixed bed reactor;
G) reducing the mixture containing the proton form of the palladium ion-laden zeolite catalyst in a hydrogen stream in the fixed bed reactor;
H) passing a flow of an inert gas through the fixed bed reactor and establishing a reaction pressure of 1 to 50 bar;
I) introducing a reactant gas feed into the fixed bed reactor comprising hydrogen, an inert gas and dimethyl ether (DME) or methanol to form a product gas mixture via a catylic reaction;
J) condensing the product gas mixture from the fixed bed reactor of step I) to a product phase liquid; and
K) separating the product phase liquid at room temperature into an aqueous phase, and into an organic phase which comprises the low-aromatics C₅₊ hydrocarbons.

### BRIEF DESCRIPTION OF THE DRAWINGS

The present invention is described in greater detail below on the basis of embodiments and of the drawings in which:

FIG. 1 shows a flow diagram of a reactor plant;

FIG. 2 shows the schematic construction of the fixed bed reactor 7 from FIG. 1;

FIG. 3 shows the DME conversion capacity of the catalyst prepared according to Example 1 as a function of DME conversion;

FIG. 4 shows the product selectivity for hydrocarbons of different chain lengths of the catalyst prepared according to Example 1 as a function of DME conversion;

FIG. 5 shows the composition of the liquid organic product phase in the chain length range C₅-C₁₁ of a catalyst prepared according to Example 1;

FIG. 6 shows the DME conversion capacity of the catalyst prepared according to Example 2 as a function of DME conversion;

FIG. 7 shows the product selectivity for hydrocarbons of different chain lengths of the catalyst prepared according to Example 2 as a function of DME conversion; and

FIG. 8 shows the composition of the liquid organic product phase in the chain length range C₅-C₁₁ of a catalyst prepared according to Example 2.

### DETAILED DESCRIPTION

The present invention provides a palladium-laden zeolite catalyst H-EU-2 having a *MRE framework structure and a process for converting DME or methanol into low-aromatics hydrocarbons in a hydrogen stream using this catalyst.

The present invention further provides a use of the aforementioned process for producing low-aromatic hydrocarbons as starting materials for a subsequent production of synthetic fuels, for example, liquefied petroleum gas, gasoline, kerosene or diesel.

The process according to the present invention comprises the following steps which is, for example, performed in the specified sequence:
A) loading an EU-2 zeolite catalyst in ammonium form with palladium ions by capillary impregnation;
B) drying and calcining the palladium ion-laden zeolite catalyst from step A) to form the proton form of the zeolite catalyst;
C) fractionating the zeolite catalyst from step B) into a catalyst powder (i.e., including granulate) having a particle size of 100 to 500 μm, for example, between 200 and 300 μm;
D) mixing the zeolite catalyst, for example, the recited catalyst powder, from step C) with an inert material, for example, in a volume ratio of catalyst to inert material of 0.1-0.2;
E) introducing the mixture from step D) into a fixed bed reactor;
F) heating the fixed bed reactor from step E) to a reaction temperature between 350° C. and 450° C., for example, between 380° C. and 400° C., and passing a flow of inert gas through the fixed bed reactor;
G) reducing the palladium ion-laden zeolite catalyst in a hydrogen stream in the fixed bed reactor;
H) passing a flow of an inert gas through the fixed bed reactor and establishing the reaction pressure of 1-50 bar;
I) introducing a reactant gas feed into the fixed bed reactor comprising hydrogen, an inert gas and DME and/or methanol, to form a product gas mixture by catalytic reaction;
J) condensing the product gas mixture from the fixed bed reactor of step I); and
K) separating the product phase liquid at room temperature into an aqueous phase and an organic phase containing C₅₊ hydrocarbons.

In an embodiment, the present invention provides a process mode which in particular relates to the aforementioned condensing of the product gas mixture according to step J) and the separating of the product phase liquid at room temperature into an aqueous phase and an organic phase containing the C₅₊ hydrocarbons according to step K). It then contains further substeps selected from the following which, in the case of a combination, can, for example, be performed in the specified sequence:
a) transferring the product gas mixture from the fixed bed reactor of step I) through a heated pipeline to at least one cold trap;
b) cooling the product gas mixture in the at least one cold trap with liquid nitrogen, wherein a product fraction containing low-aromatics C₅₊ hydrocarbons is frozen to afford a solid product fraction;
c) collecting the solid product fraction in a collection vessel of the cold trap;
d) thawing the solid product fraction to afford a liquid product fraction and subsequently withdrawing the liquid product fraction from the collection vessel of the cold trap of step (c) and separating the aqueous from the organic product phase; and
e) analyzing the organic product phase from step d) in an external gas chromatograph.

In a first step A) of the process according to the present invention, a zeolite catalyst EU-2 in the ammonium form is loaded with palladium ions. The palladium ions are applied by capillary impregnation (incipient wetness impregnation), wherein a low palladium content having a mass fraction w_{Palladium} of 0.01-1%, based on the total catalyst mass, is established. In an embodiment, the mass fraction w_{palladium}=0.35%.

In the process according to the present invention, capillary impregnation (incipient wetness impregnation) is to be understood as meaning that an aqueous solution of a palladium salt, as a precursor of hydrogenation-active palladium(0), is applied to the zeolite catalyst EU-2 which has a pore volume identical to the volume of the solution added. The solution is drawn into the pores by capillary action.

In the process according to the present invention, the zeolite catalyst has an Si/Al amount of substance ratio r_{Si}/Al of, for example, 50-100 mol·mol⁻¹. The amount of substance ratio rᵢⱼ is defined as the value of the quotient of the amount of substance nᵢ of the one mixture component i under consideration and the amount of substance nⱼ of the other mixture component j under consideration (standard DIN 1310: composition of mixed phases).

The catalyst is dried and calcined in a next step B) to expel the volatile constituents in the solution and to deposit the palladium salt on the catalyst surface. This converts the EU-2 catalyst from the ammonium form into the proton form H-EU-2 by liberation of NH₃.

In a further step C), the zeolite catalyst is fractionated to a particle size of 100 to 500 μm, for example, to a particle size of 224 to 300 μm.

In an embodiment, the fractionated zeolite catalyst from step C) can, for example, be converted into extrudates, pellets or other shaped bodies in the course of a catalyst shaping (S. Devyatkov et al., Chimica Oggi—Chemistry Today 2015, 33, 6, 57-64; R. Bingre et al., Catalysts 2018, 8, 163). This can, for example, be effected before the mixing in step D). Compared to a catalyst powder, extrudates, pellets or other shaped articles, especially in an industrial production process, are easier to handle and meter and are substantially more stable. The mixing of the zeolite catalyst with an inert material in step D) is then carried out not with the catalyst powder, but rather with the preformed catalyst.

In the following step D) of the process according to the present invention, the zeolite catalyst from step C) is mixed with an inert material, wherein the volume ratio of catalyst to inert material is 0.1-0.2. The volume ratio ψᵢⱼ is defined as the value of the quotient of the volume Vi of the one mixture component i under consideration and the volume Vⱼ of the other mixture component j under consideration (standard DIN 1310: composition of mixed phases).

Suitable inert materials in the process according to the present invention can, for example, include silicon carbide, quartz glass (sand or pearls), α-alumina or other technical ceramics.

The process according to the present invention can, for example, employ silicon carbide having a particle size of 10 to 500 μm, for example, having a particle size of 100 to 180 μm.

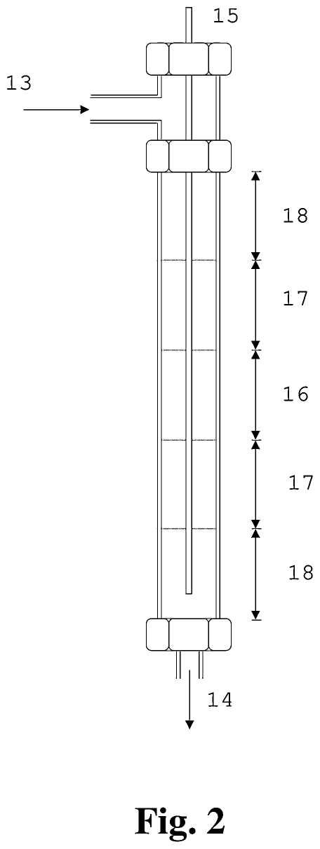

In step E) of the process according to the present invention, the mixture from step D) is introduced into a fixed bed reactor and forms the reaction zone in the fixed bed reactor (see FIG. 2). In an embodiment, this reaction zone can, for example, be flanked by an inflow zone and an outflow zone which is composed of the aforementioned inert material. In the process according to the present invention, the inert material is used for catalyst stabilization. Since the conversion of DME or methanol into low-aromatics hydrocarbons in the process according to the present invention is an exothermic reaction, the inert material provides a virtually isothermal temperature profile in the reaction zone. The process according to the present invention can, for example, employ silicon carbide as inert material in the inlet zone and the outlet zone.

In step F) of the process according to the present invention, the fixed bed reactor is heated to the reaction temperature and a flow of inert gas is passed therethrough. The reaction temperature in the process according to the present invention is 350-450° C., for example, 380-400° C.

In the process according to the present invention, it has been found that a reaction temperature of at least 380° C. is advantageous for formation of longer-chain hydrocarbons at high reactant conversion.

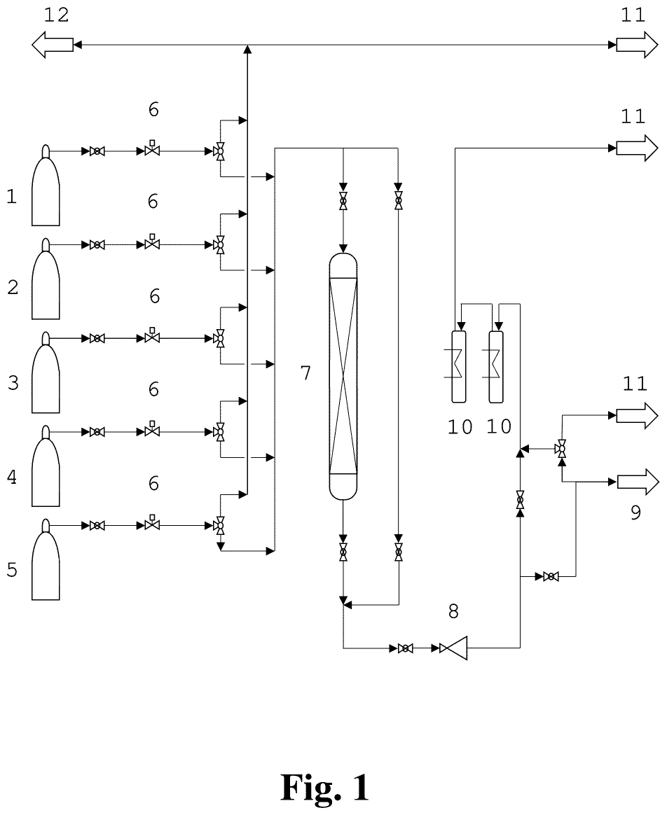

The employed inert gas can, for example, be N₂, He or Ar. The inert gas can, for example, be N₂ (see FIG. 1).

In step G) of the process according to the present invention, the palladium ion-laden zeolite catalyst is reduced in a hydrogen stream in the fixed bed reactor, thus producing a zeolite catalyst containing hydrogenation-active palladium(0).

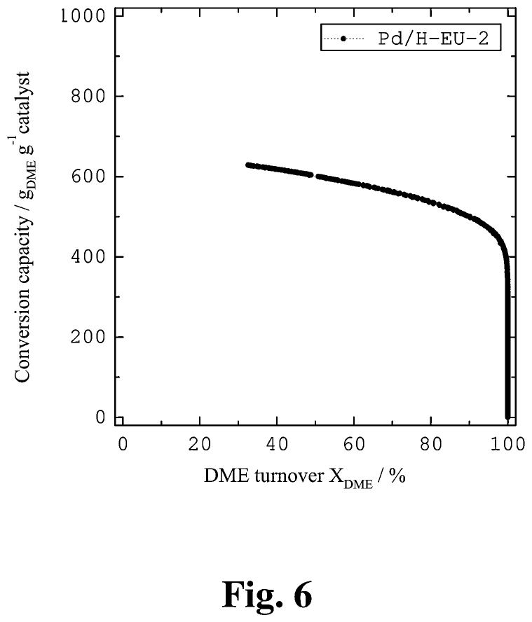

In step H) of the process according to the present invention, a flow of inert gas is again passed through the fixed bed reactor while simultaneously establishing the reaction pressure. The inert gas can, for example, be selected from N₂, He or Ar. In an embodiment, the inert gas is N₂. The reaction pressure in the process according to the present invention is 1-50 bar, for example, 1-30 bar. It has been found in the process according to the present invention that a reaction pressure of 20-30 bar provides for particularly high reaction conversions (see FIG. 6).

In step I) of the process according to the present invention, a reactant gas feed is introduced into the fixed bed reactor. The reactant gas feed includes hydrogen, an inert gas and DME or methanol. In an embodiment, the volume fraction in the reactant gas feed φ_{DME or methanol} is 1-10%, for example, 5%. The volume fraction of hydrogen in the reactant gas feed φ_{hydrogen} in the process according to the present invention is 1-50%, for example, 10-30%. The volume fraction φᵢ is defined as the value of the quotient of the volume Vᵢ of a mixture component i under consideration and the total volume V₀ before the mixing operation. The latter is the sum of the starting volumes of all mixture components (including i) of the mixture (Standard DIN 1310: composition of mixed phases).

In the process according to the present invention, it has been found that, in the reactant gas feed, a volume ratio ψ_{H2/DME or methanol} of hydrogen to DME or methanol of 4 results in particularly high reaction conversions and relatively high long-term stability of the catalyst (see FIG. 6).

In the process according to the present invention, the reaction of DME or methanol is carried out at a weight hourly space velocity (WHSV) of 0.1-20 g_{DME or methanol}·g_{catalyst}⁻¹·h⁻¹, for example, at a WHSV of 1-5 g_{DME or methanol}·g_{catalyst}⁻¹·h⁻¹.

After the forming of the product gas mixture in step I) and before the condensing in step J), the product gas mixture can, for example, be transferred from the fixed bed reactor through a heated pipe conduit to at least one cold trap in which the condensation is carried out. In the course of this transferring, at least a substream of the product gas mixture (for example, diverted from the heated pipe conduit) can, for example, be diverted into an online gas chromatograph for continuous determination of the DME or methanol conversion and the composition of the product gas mixture and subsequently passed to the cold trap (either directly or via a return to the heated pipe conduit).

In step J) of the process according to the present invention, the product gas mixture formed in the aforementioned step I) is condensed. In step K), the product phase liquid at room temperature is then separated into an aqueous phase and an organic phase containing C₅₊ hydrocarbons.

This can, for example, comprise a transferring of the product gas mixture from the fixed bed reactor to the condensation, for example, into at least one cold trap through a heated pipe conduit to the at least one cold trap. The transferring can, for example, comprise diverting the product gas mixture, for example, at least a substream of the product gas mixture, through an online gas chromatograph. During this optional complete or at least partial diverting of the product gas stream, the online (i.e., real-time) gas chromatograph continuously monitors the DME or methanol conversion and the composition of the product gas mixture.

The product gas mixture can, for example, also be cooled with liquid nitrogen in at least one cold trap after the optional online gas chromatography analysis. The product fraction containing low-aromatics C₅₊ hydrocarbons can, for example, be frozen and collected in a collection vessel of the cold trap. In a process embodiment, the frozen product fraction can then, for example, be thawed and withdrawn from the collection vessel of the cold trap as a liquid product and the organic product phase separated from the aqueous phase in a phase separator. In an embodiment, the organic product phase can then, for example, be analyzed in an external gas chromatograph, for example, according to the specifications of the standards EN ISO 22854 or ASTM D6839 (see FIG. 1).

The process according to the present invention has the following advantages:

The organic product mixture (gasoline) in the chain length range C₅-C₁₁ is gasoline-like and features a comparatively low aromatics content φ_{aromatics} of not more than about 3%. Analyses of the paraffins and olefins thus obtained indicate a comparatively high degree of molecular branching. This indicates a good fuel compatibility of the organic product phase (compare Tables 1 and 2).

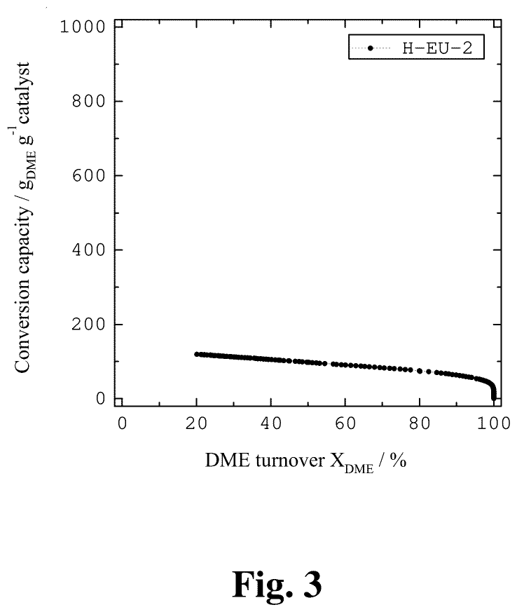

In addition to optimizing the product spectrum, the process according to the present invention exhibits a high catalyst activity, high conversions, high space-time yields, and a huge increase in catalyst stability and lifetime. The palladium-laden H-EU-2 catalyst exhibits a high long-term stability even at high temperatures. The long-term stability makes it possible to achieve high conversions and high selectivities over periods of several days to several weeks (compare FIGS. 3 and 6).

In the process according to the present invention, the loading of the zeolite with palladium leads to a very high stability of the catalyst to sintering, thus also allowing for selection of relatively high reaction temperatures of around 400° C. This results in high catalyst activity and high DME/methanol conversion. The addition of hydrogen not only favorably influences the product spectrum in the process according to the present invention, but also increases the long-term stability of the catalyst system compared to the prior art.

These effects are surprising since the one-dimensional pore system of the zeolite EU-2 should promote rapid catalyst deactivation in the DTG/MTG reaction due to the blocking of the pore channels by large molecules, for example, methylated aromatics. Although the product mixture contains small amounts of methylated aromatics and higher hydrocarbons, rapid catalyst deactivation is not observed in the process according to the present invention. The use of the hydrogenation catalyst Pd(0) in conjunction with hydrogen should furthermore suppress the formation of longer hydrocarbon chains in the process according to the present invention and increase the formation of low molecular weight hydrocarbons, for example, methane, ethylene or ethane. These are undesirable byproducts of the DTG/MTG reaction and are formed only to a subordinate degree according to the present invention.

The process according to the present invention may be used to produce synthetic fuels, for example, liquefied petroleum gas, gasoline, kerosene or diesel. Applications as solvents and in the production of chemicals are likewise possible.

The present invention is more particularly elucidated with reference to the following figures, working examples and descriptions. All specified features and combinations thereof are thereby not limited only to these figures and working examples and their embodiments. On the contrary, these are to be regarded as combinable as representatives of further embodiments which are possible but not explicitly specified as working examples.

FIG. 1 shows a flow diagram of a reactor plant. The reactor plant comprises the feeding regions of reactant and inert gases 1-5, whose flows are adjusted using appropriate mass flow controllers 6. Arranged downstream thereof is the fixed bed reactor 7 which is coupled to a pressure control valve 8. Arranged downstream thereof are apparatuses for product workup composed substantially of at least one cold trap 10 which cools the product gas mixture with liquid nitrogen. Analysis of the product mixtures is effected using an online gas chromatograph 9 which is operated using a substream continuously withdrawn from the product stream. Further elements include a flowmeter 12 and an apparatus for discharging gaseous components (offgas 11).

FIG. 2 shows the schematic construction of the fixed bed reactor 7 from FIG. 1 comprising an inlet 13 for the reactant gas feed and an outlet 14 for the reaction products. The catalyst powder was fractionated to a particle size dp in the range from 224 to 300 μm. The samples were subsequently mixed with inert silicon carbide (SiC, particle size in the range from 100 to 180 μm) (0.333 g of catalyst in SiC in a volume ratio ψcatalyst/SiC=0.1) to prevent a temperature elevation in the catalyst bed as a result of the exothermic DTG reaction and to provide a virtually isothermal temperature profile in the reaction zone (ΔTaxial<2 K). The catalyst/SiC mixture formed the reaction zone 16 in a fixed bed reactor made of stainless steel (internal diameter di=12 mm). The reaction zone 16 was flanked by an inlet zone and an outlet zone 17 composed of inert SiC, which were secured in the reaction tube by inert glass wool 18. The reactor was heated using three heating blocks. Two thermocouples were installed in each heating block to control and monitor the temperature. The axial temperature profile in the reaction zone 16 was further recordable via a thermocouple in the tube reactor movable within a guide sleeve 15.

FIG. 3 shows the DME conversion capacity of the catalyst prepared according to Example 1 as a function of DME conversion.

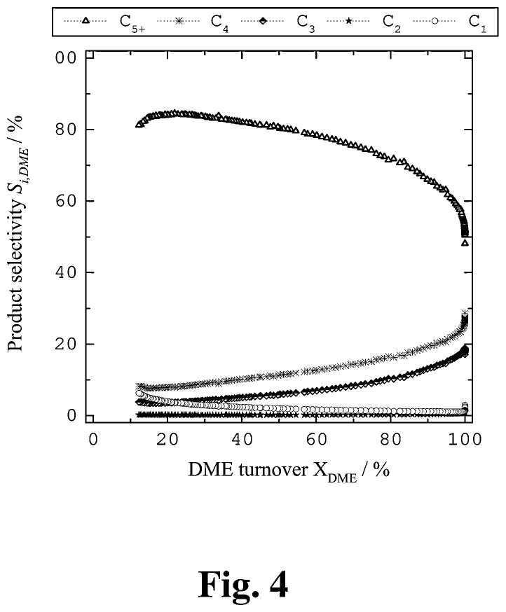

FIG. 4 shows the product selectivity for hydrocarbons of different chain lengths of the catalyst prepared according to Example 1 as a function of DME conversion.

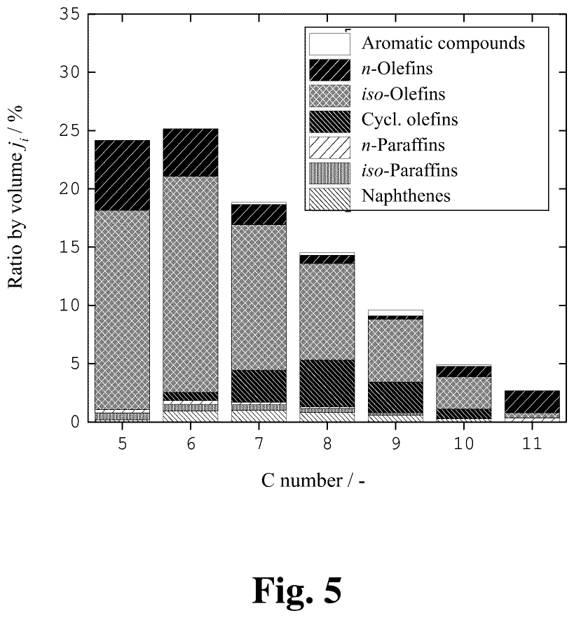

FIG. 5 shows the composition of the liquid organic product phase in the chain length range C₅-C₁₁ of a catalyst prepared according to Example 1. Sampling was carried out from commencement of the experiment until reduction of the conversion to X_{DME}=40%. Analysis was carried out according to the specifications of the standards EN ISO 22854 or ASTM D6839.

FIG. 6 shows the DME conversion capacity of the catalyst prepared according to Example 2 as a function of DME conversion.

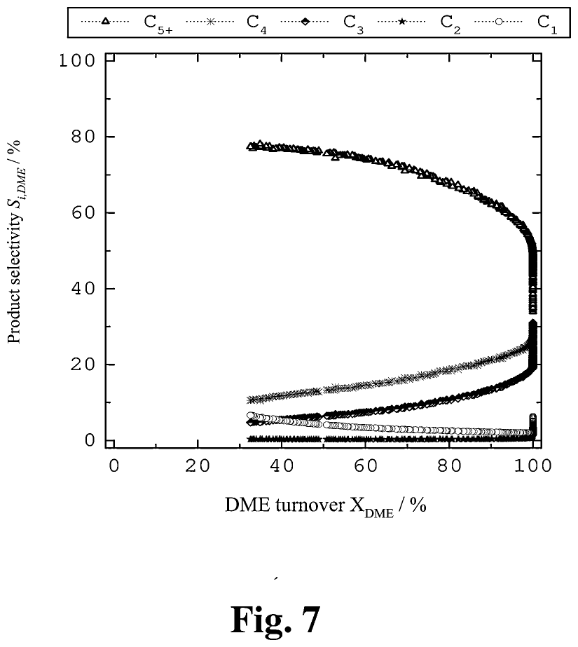

FIG. 7 shows the product selectivity for hydrocarbons of different chain lengths of the catalyst prepared according to Example 2 as a function of DME conversion.

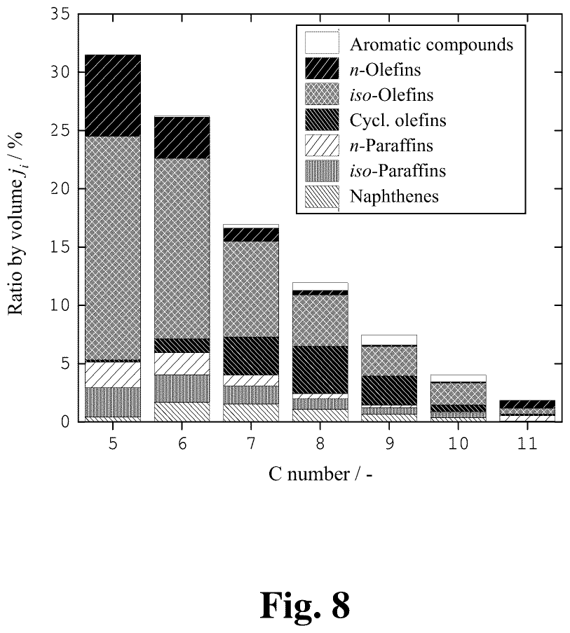

FIG. 8 shows the composition of the liquid organic product phase in the chain length range C₅-C₁₁ of a catalyst prepared according to Example 2. Sampling was carried out from commencement of the experiment until reduction of the conversion to X_{DME}=40%. Analysis was carried out according to the specifications of the standards EN ISO 22854 or ASTM D6839.

### EXAMPLES

### Example 1

### Catalyst Preparation Reference Material

Zeolite EU-2 of structure type *MRE was used as the starting material for the investigations. The EU-2 powder having an Si/Al ratio of r_{Si/Al}=78 mol·mol⁻¹ and a specific surface area of 256 m²·g⁻¹ was provided in the ammonium form (NH₄ form) and was initially freed of potential impurities by an ion exchange process (3 ion exchanges in a 1M NH₄NO₃ washing solution at 75° C. for 2 h in each case). The powder was then dried overnight in a vacuum drying cabinet at 80° C. and 10 mbar. The dry powder was then heated to a temperature of 550° C. in a calcining furnace at a heating rate of 2 K·min⁻¹ and calcined for 6 h at this temperature. The NH₄ form of the zeolite powder was converted into the proton form (H form, H zeolite). The zeolite catalyst H-EU-2 is used as a reference material to demonstrate the advantages of a metal-laden catalyst in a process for converting DME or methanol into low-aromatics hydrocarbons in a hydrogen stream using this catalyst.

### Example 2

### Catalyst Preparation of Metal-Laden Zeolite

To produce metal-laden zeolite catalysts, zeolite EU-2 of structure type *MRE having the same material properties as in Example 1 was used. After 3 ion exchanges (1M NH₄NO₃ washing solution at 75° C. for 2 h in each case), the powder in the ammonium form was dried overnight in a vacuum drying cabinet at 80° C. and 10 mbar. Loading with palladium was then carried out by “incipient witness impregnation”. The EU-2 powder was impregnated dropwise with a solution of Pd(NO₃)₂(NH₃)₄xH₂O in deionized water. The resulting paste was then dried overnight in a drying oven at 40° C. The dry powder was then heated to a temperature of 550° C. in a calcining furnace at a heating rate of 2 K·min⁻¹ and calcined for 6 h at this temperature. The NH₄ form of the zeolite was converted into the proton form (H form, H zeolite).

### Example 3

### Conversion of DME

The catalyst powder was fractionated to a particle size dₚ in the range from 224 to 300 μm. The samples were subsequently diluted with inert silicon carbide (SiC, particle size in the range from 100 to 180 μm) in a mixing ratio of ψ_{catalyst/SiC}=0.1 to prevent a temperature elevation in the catalyst bed as a result of the exothermic DTG reaction and to provide a virtually isothermal temperature profile in the reaction zone (ΔTₐₓᵢₐₗ<2 K). The catalyst/SiC mixture formed the reaction zone 16 in a fixed bed reactor made of stainless steel (internal diameter dᵢ=12 mm). The reaction zone 16 was flanked by an inlet zone and an outlet zone 17 composed of inert SiC, which were secured in the reaction tube by inert glass wool 18. The construction of the fixed bed reactor is schematically illustrated in FIG. 2. The reactor was heated by a heating jacket composed of three aluminum blocks, each comprising four tight-fitting heating cartridges. During the heating process, the reactor with a flow of 80 mlₙ·min⁻¹ of N₂ was passed through the reactor.

The heating blocks were thermally insulated by a vermiculite jacket. This minimized external heat loss and provided a uniform heat distribution in the reactor coupled with good reliability. Two thermocouples were installed in each heating block to control and monitor the temperature. The axial temperature profile in the reaction zone 16 was further recordable via a thermocouple in the tube reactor movable within a guide sleeve 15. Heating of the fixed bed reactor to reaction temperature (400° C.) was followed by reduction of the sample in the case of the metal-laden zeolite catalyst (Pd/H-EU-2). A flow of 80 mlₙ·min⁻¹ of H₂ was passed through the reactor for 5 h. This step was omitted in the case of the reference sample without metal loading (H-EU-2).

Following the reduction, a flow of 80 mlₙ·min⁻¹ of N₂ was passed through the reactor while the reaction pressure was adjusted via a manual supply pressure control valve (1.5-30 bar). The reaction investigation of the metal-laden zeolite catalyst (Pd/H-EU-2) was then carried out by passing a reactant gas feed consisting of φ_{DME}=5% and also N₂ and H₂ in varying proportions into the reactor. The reactant gas stream was selected so that the experiments were performed at a WHSV of 1.5 g_{DME}·g_{catalyst}⁻¹·h⁻¹ unless otherwise stated.

For analysis, the reaction products were passed through a pipe conduit heated to 180° C. to an online gas chromatograph (for example, Agilent 7890B) fitted with an FID (flame ionization detector) for determining hydrocarbons/oxygenates and a TCD (thermal conductivity detector) for determining permanent gases/water. Assignment of the peaks of hydrocarbons in the range C₁-C₄, the oxygenates methanol and DME and the permanent gases H₂, N₂, CO₂ and CO was undertaken with the aid of calibration gases from Air Liquide. The further peaks of the FID spectrum were combined into the group C₅₊ in the online analysis. The DME conversion X_{DME} of the samples was calculated via the ratio of the reacted DME amount (difference between inflowing DME stream N_{DME,in} and the outflowing DME stream N_{DME,out}) to the DME inflow stream according to equation (I):

$${X}_{D M E} = \frac{{\dot{N}}_{DME , in} - {\dot{N}}_{DME , out}}{{\dot{N}}_{DME , in}} \cdot 100 \% \qquad ( I )$$

The conversion capacity of a catalyst describes the cumulatively converted mass of a reactant based on the mass of the catalyst m_{cat} as a function of the time-on-stream (TOS). Plotting the conversion capacity above the conversion allows for comparability of the deactivation behavior in long-term measurements performed with different WHSV values. The conversion capacity is derived according to equation (II) from the product of the WHSV and the integral area under a corresponding conversion-TOS curve:

$$conversion capacity = \frac{{m}_{DME}}{{m}_{cat}} = WHSV \cdot {\int }_{0}^{T O S} {X}_{D M E} dTOS \qquad ( II )$$

The selectivities S of the products i formed from DME (S_{i,DME}) was formed by integration of the areas of the respective GC-FID signals Iᵢ relative to the total GC-FID area Iₜₒₜₐₗ minus the DME signal I_{DME} and the methanol signal Iₘₑₜₕₐₙₒₗ. Methanol is in equilibrium with DME via the dehydration/hydration reaction and is considered a reactant of the MTG reaction which is analogous to the DTG reaction. The selectivities for the carbon-containing (by) products CO and CO₂ were also subtracted from the quotient of the signal intensities. These were not captured by the GC-FID signal, but were determinable from the GC-TCD spectrum according to equation (III) using a multi-point calibration with calibration gases from Air Liquide:

$${S}_{i , DME} = \frac{{v}_{i}}{{v}_{DME}} \cdot \frac{{\dot{N}}_{i , out}}{{\dot{N}}_{DME , in} - {\dot{N}}_{DME , out}} \cdot 100 \% \qquad ( III )$$

The selectivity S_{i,DME} for the hydrocarbon products was thus determined according to equation (IV):

$${S}_{i , DME} = \frac{{I}_{i}}{{I}_{total} - {I}_{DME} - {I}_{Methanol}} \cdot ( 100 \% - {S}_{CO , DME} - {S}_{CO 2 , DME} ) \qquad ( IV )$$

For more precise determination of the products in the chain length range C₅₊, a cold trap for condensation/freezing of these components was arranged downstream of the online gas chromatograph. The liquid organic condensate in the cooling trap was analyzed after the end of the experiment in an M4 reformulyzer (from PAC). A flow diagram of the experimental apparatus for reaction investigation of the conversion of DME over metal-laden H-EU-2 catalysts is shown in FIG. 1.

### Example 4

In Example 4, the catalytic performance of an H-EU-2 catalyst produced by the preparation method specified in Example 1 was tested. The reaction described in this example was performed at 400° C., 20 bar of reaction pressure, and a WHSV of 1.5 g_{DME}·g_{catalyst}⁻¹·h⁻¹ at a constant volume fraction φ_{DME}=5% in N₂. The DME conversion capacity of the catalyst is plotted above the DME conversion X_{DME} in FIG. 3. The conversion capacity of the H-EU-2 catalyst is used as a reference for classifying the advantages in terms of activity and long-term stability of a metal-laden catalyst in a process for converting DME or methanol into low-aromatics hydrocarbons in a hydrogen stream which uses the catalyst.

### Example 5

In Example 5, the product selectivity of an H-EU-2 zeolite catalyst produced according to Example 1 in a reaction at 400° C., 20 bar of reaction pressure and a WHSV of 1.5 g_{DME}·g_{catalyst}⁻¹·h⁻¹ at a constant volume fraction φ_{DME}=5% in N₂ was determined. FIG. 4 shows the selectivity in terms of the chain length of the resulting hydrocarbons (C₁, C₂, C₃, C₄ and C₅₊) above the DME conversion X_{DME} as determined with the online gas chromatograph. Hydrocarbons in the C₅₊ chain length range were the desired product components. The proportion of the product fraction C₅₊ was in the range from 45-85% over the total experimental duration.

### Example 6

In Example 6, the composition of the liquid product phase from the catalytic conversion of DME over an H-EU-2 catalyst prepared according to Example 1 was determined. The experimental procedure was carried out as described in Example 3 at a temperature of 400° C., a WHSV of 1.5 g_{DME}·g_{catalyst}⁻¹·h⁻¹ at a reaction pressure of 20 bar, and a constant volume fraction φ_{DME}=5% in N₂. The sample for analysis of the liquid organic product phase (C₅-C₁₁) was generated in the time window from reaction commencement until a decrease in DME conversion to X_{DME}=40%. The analysis was carried out according to the specifications of the standards EN ISO 22854 and ASTM D6839 and the volume fractions of the products are shown graphically in FIG. 5 as a function of carbon number and listed numerically in Table 1. A high olefin content was found for all chain lengths, wherein branched isomers predominate markedly. Paraffins and aromatics were present only in small amounts. The volume fraction of the aromatics was below 2%.

### Example 7

In Example 7, the catalytic performance of a Pd/H-EU-2 catalyst produced by the preparation method specified in Example 2 was tested. The reaction described in this example was performed at 400° C., 20 bar of reaction pressure and a WHSV of 1.5 g_{DME}·g_{catalyst}⁻¹·h⁻¹ at constant volume fractions φ_{DME}=5%, φ_{H2}=20% and φ_{N2}=75%. The DME conversion capacity of the catalyst Pd/H-EU-2 is plotted above the DME conversion X_{DME} in FIG. 6. In a process for converting DME or methanol into low-aromatics hydrocarbons in a hydrogen stream, the conversion capacity of the catalyst Pd/H-EU-2 is increased more than five-fold compared to the reference system in Example 4. This implies a markedly elevated activity and stability of the catalyst.

### Example 8

In Example 8, the product selectivity of the Pd/H-EU-2 catalyst produced according to Example 2 in a reaction at 400° C., 20 bar of reaction pressure, and a WHSV of 1.5 g_{DME}·g_{catalyst}⁻¹·h⁻¹ at constant volume fractions φ_{DME}=5%, φ_{H2}=20% and φ_{N2}=75% was determined. FIG. 7 shows the selectivity in terms of the chain length of the resulting hydrocarbons (C₁, C₂, C₃, C₄ and C₅₊) above the DME conversion X_{DME} as determined with the online gas chromatograph. Hydrocarbons in the C₅₊ chain length range were the desired product components. The proportion of the product fraction C₅₊ was in the range from 35-80% over the total experimental duration. The excellent selectivity for the C₅₊ product group of the reference sample from Example 5 is reduced only slightly by the particular embodiment of a Pd/H-EU-2 catalyst in a process for reacting DME or methanol to low-aromatic hydrocarbons in a hydrogen stream.

### Example 9

In Example 9, the composition of the liquid product phase from the catalytic conversion of DME over a Pd/H-EU-2 catalyst prepared according to Example 2 was determined. The experimental procedure was carried out as described in Example 3 at a temperature of 400° C., a WHSV of 1.5 g_{DME}·g_{catalyst}⁻¹·h⁻¹ at a reaction pressure of 20 bar, and constant volume fractions φ_{DME}=5%, φ_{H2}=20% and φ_{N2}=75%. The analysis was carried out according to the specifications of the standards EN ISO 22854 and ASTM D6839 and the volume fractions of the products are shown graphically in FIG. 8 as a function of carbon number and listed numerically in Table 2. Analogous to the reference sample from Example 6, a high olefin content was found for all chain lengths. Branched isomers likewise predominate markedly. In a process for converting DME or methanol into low-aromatics hydrocarbons in a hydrogen stream, the paraffin fraction is slightly elevated in the embodiment of a Pd/H-EU-2 catalyst compared to the reference from Example 6. The volume fraction of the aromatics is in turn low and is below 3%.

Table 1 shows the numerical values of the gas chromatographic analysis of the liquid organic product phase (C₅-C₁₁) which was generated in the time window from reaction commencement (initial DME conversion X_{DME} of 100%) until a decrease in DME conversion to X_{DME}=40% (production of the catalyst according to Example 1). The analysis was performed according to the standards EN ISO 22854 and ASTM D6839 and showed a very low volume fraction of aromatics in the sample of 1.1% and a very high volume fraction of olefins of more than 90%. The volume ratio of branched olefins to linear olefins was high and amounted to 4.2.

Table 2 shows the numerical values of the gas chromatographic analysis of the liquid organic product phase (C₅-C₁₁) which was generated in the time window from reaction commencement (initial DME conversion X_{DME} of 100%) until a decrease in DME conversion to X_{DME}=40% (production of the catalyst according to Example 2). The analysis was carried according to the standards EN ISO 22854 and ASTM D6839 and showed a very low volume fraction of aromatics of 2.6% and a high volume fraction of olefins of 77%. The volume ratio of branched olefins to linear olefins was high and amounted to 4.1.

TABLE 1

| Carbon | Total | n-paraffins | i-paraffins | Naphthenes | n-olefins | i-olefins | Cyclic | Aromatics |
|---|---|---|---|---|---|---|---|---|
| number | (%) | (%) | (%) | (%) | (%) | (%) | olefins (%) | (%) |
| 5 | 24.2 | 0.3 | 0.6 | 0.2 | 6.0 | 17.1 | — | — |
| 6 | 25.2 | 0.3 | 0.6 | 0.9 | 4.1 | 18.6 | 0.7 | — |
| 7 | 18.9 | 0.2 | 0.5 | 1.0 | 1.7 | 12.5 | 2.7 | 0.2 |
| 8 | 14.6 | 0.1 | 0.3 | 0.8 | 0.7 | 8.3 | 4.0 | 0.3 |
| 9 | 9.6 | — | 0.2 | 0.6 | 0.3 | 5.4 | 2.6 | 0.5 |
| 10 | 4.9 | — | — | 0.3 | 0.9 | 2.8 | 0.8 | 0.1 |
| 11 | 2.7 | 0.4 | — | — | 1.9 | 0.4 | — | — |
| Total | 100 | 1.3 | 2.2 | 3.8 | 15.6 | 65.1 | 10.9 | 1.1 |

TABLE 2

| Carbon | Total | n-paraffins | i-paraffins | Naphthenes | n-olefins | i-olefins | Cyclic | Aromatics |
|---|---|---|---|---|---|---|---|---|
| number | (%) | (%) | (%) | (%) | (%) | (%) | olefins (%) | (%) |
| 5 | 31.5 | 2.2 | 2.5 | 0.4 | 7.0 | 19.2 | 0.2 | — |
| 6 | 26.2 | 1.9 | 2.3 | 1.7 | 3.5 | 15.5 | 1.2 | 0.1 |
| 7 | 17.0 | 0.9 | 1.5 | 1.5 | 1.1 | 8.2 | 3.3 | 0.3 |
| 8 | 11.9 | 0.5 | 0.9 | 1.1 | 0.4 | 4.4 | 4.0 | 0.7 |
| 9 | 7.5 | 0.2 | 0.5 | 0.7 | 0.1 | 2.6 | 2.5 | 0.9 |
| 10 | 4.0 | 0.1 | 0.4 | 0.4 | 0.1 | 1.9 | 0.6 | 0.6 |
| 11 | 1.8 | 0.5 | 0.1 | — | 0.7 | 0.5 | 0.4 | — |
| Total | 100 | 6.3 | 8.3 | 5.8 | 12.9 | 52.3 | 11.8 | 2.6 |

### LIST OF REFERENCE NUMERALS

1 DME/N₂ or Methanol/N₂ reactant gas mixture
2 Hydrogen
3 N₂ inert gas
4 He inert gas
5 Ar inert gas
6 Mass flow controllers
7 Fixed bed reactor
8 Pressure control valve
9 Online gas chromatograph
10 Cold trap
11 Offgas
12 Flowmeter
13 Inlet
14 Outlet
15 Guide sleeve for axial thermocouple
16 Reaction zone with catalyst and SiC
17 Inlet or outlet zone
18 Glass wool

1-16. (canceled)

17. A method for converting dimethyl ether (DME) or methanol into low-aromatics C₅₊ hydrocarbons via a catalytic reaction over a palladium-laden zeolite catalyst H-EU-2 in a hydrogen stream, the method comprising:
A) loading a EU-2 zeolite catalyst in an ammonium form with palladium ions via a capillary impregnation to obtain a palladium ion-laden zeolite catalyst;
B) drying and calcining the palladium ion-laden zeolite catalyst from step A) to form a proton form of the palladium ion-laden zeolite catalyst;
C) fractionating the proton form of the palladium ion-laden zeolite catalyst from step B) into a powder having a particle size of 100 to 500 μm;
D) mixing the powder from step C) with an inert material to obtain a mixture;
E) introducing the mixture from step D) into a fixed bed reactor;
F) heating the fixed bed reactor from step E) to a reaction temperature of 350 to 450° C. and passing a flow of an inert gas through the fixed bed reactor;
G) reducing the mixture containing the proton form of the palladium ion-laden zeolite catalyst in a hydrogen stream in the fixed bed reactor;
H) passing a flow of an inert gas through the fixed bed reactor and establishing a reaction pressure of 1 to 50 bar;
I) introducing a reactant gas feed into the fixed bed reactor comprising hydrogen, an inert gas and dimethyl ether (DME) or methanol to form a product gas mixture via a catylic reaction;
J) condensing the product gas mixture from the fixed bed reactor of step I) to a product phase liquid; and
K) separating the product phase liquid at room temperature into an aqueous phase, and into an organic phase which comprises the low-aromatics C₅₊ hydrocarbons.

18. The process as recited in claim 17, wherein the palladium ion-laden zeolite catalyst in step A) has a palladium loading having a mass fraction w_{palladium}, based on a total catalyst mass, of 0.01 to 1%.

19. The process as recited in claim 17, wherein the inert material in step D) is silicon carbide, quartz glass, α-aluminum oxide or a technical ceramic.

20. The process as recited in claim 17, wherein the EU-2 zeolite catalyst in step A) has an Si/Al substance ratio r_{Si/Al} of 50 to 100 mol·mol⁻¹.

21. The process as recited in claim 17, wherein the converting of the dimethyl ether (DME) or the methanol via the catylic reaction in step I) is performed with a volume fraction of the dimethyl ether (DME) or the methanol in the reactant gas feed of φ_{DME or methanol}=1 to 10%.

22. The process as recited in claim 17, wherein the converting of the dimethyl ether (DME) or the methanol via the catylic reaction in step I) is performed with a volume fraction of the hydrogen in the reactant gas feed of φ_{hydrogen}=1 to 50%.

23. The process as recited in claim 17, wherein the catalytic reaction of the dimethyl ether (DME) or the methanol in step I) is performed at a weight-hourly space velocity of 1 to 10 g_{DME or methanol}·g_{catalyst}⁻¹·h⁻¹.

24. The process as recited in claim 17, wherein a product fraction of the low-aromatics C₅₊-hydrocarbons is at least 35%.

25. The process as recited in claim 24, wherein a volume fraction of aromatics in the product fraction of the low-aromatics C₅₊ hydrocarbons under reaction conditions which allow for an initial dimethyl ether (DME) conversion X_{DME} of 100% for an time-on-stream from X_{DME}=100% to X_{DME}=40% is below 3%.

26. The process as recited in claim 25, wherein under the reaction conditions which allow for the initial dimethyl ether (DME) conversion X_{DME} of 100%, a volume fraction of branched olefins in the product fraction of the low-aromatics C₅₊ hydrocarbons is at least 50% for the time-on-stream from X_{DME}=100% to X_{DME}=40%, and a volume ratio of the branched olefins to linear olefins is at least 4.

27. The process as recited in claim 17, further comprising:
transferring the product gas mixture from the fixed bed reactor in step I) through a heated pipe conduit to at least one cold trap; and
preforming the condensing of the product gas mixture from the fixed bed reactor of step I) in step J) in the at least one cold trap.

28. The process as recited in claim 27, further comprising:
during the transferring, diverting at least a substream of the product gas mixture into an online gas chromatograph;
continually providing a determination of a dimethyl ether (DME) conversion or a methanol conversion and a composition of the product gas mixture via the online gas chromatograph; and then transferring the substream to the at least one cold trap.

29. The process as recited in claim 17, further comprising:
converting the powder from step C) into extrudates or pellets prior to mixing the powder from step C) with the inert material to obtain the mixture in step D).

30. The process as recited in claim 17, wherein the mixing of the powder from step C) with the inert material to obtain the mixture in step D is performed with a volume ratio of the proton form of the palladium ion-laden zeolite catalyst to the inert material of 0.1 to 0.2;

31. The process as recited in claim 17, wherein the condensing of the product gas mixture from the fixed bed reactor of step I) and the separating the product phase liquid at room temperature into the aqueous phase and the organic phase which comprises the low-aromatics C₅₊ hydrocarbons of step J), comprises:
a) transferring the product gas mixture from the fixed bed reactor of step I) through a heated pipeline to at least one cold trap;
b) cooling the product gas mixture in the at least one cold trap with liquid nitrogen, wherein a product fraction containing the low-aromatics C₅₊ hydrocarbons is frozen to afford a solid product fraction;
c) collecting the solid product fraction in a collection vessel of the at least one cold trap;
d) thawing the solid product fraction collected in the collection vessel to provide a liquid product fraction and then withdrawing the liquid product fraction from the collection vessel of the at least one cold trap of step c) and separating the aqueous phase from the organic phase; and
e) analyzing the organic phase from step d) in an external gas chromatograph.

32. A method of using the process as recited in claim 17 for producing low-aromatics C₅₊ hydrocarbons, the method comprising:
providing the low-aromatics C₅₊ hydrocarbons via the method as recited in claim 17; and
using the low-aromatics C₅₊ hydrocarbons as a starting material for a subsequent production of a synthetic fuel.

33. The method as recited in claim 32, where in the synthetic fuel is liquefied petroleum gas, gasoline, kerosene or diesel.
